SEQUENCE LISTING (15 sequences)

SEQ ID NO: 1 - protein, 1042 aa, Artificial sequence
  PEPTIDE (1)..(1042): PDE11GAF/CyaB1-Chimera
MAASRLDFGEVETFLDRHPELFEDYLMRKGKQEMVEKWLQRHSQGQGALGPRPSLAGTSSLAHSTCRGGSSVGGGTGPNGSAHSQPLPGGGDCGGVPLSPSWAGGSRGDGNLQRRASQKELRKSFARSKAIHVNRTYDEQVTSRAQEPLSSVRRRALLRKASSLPPTTAHILSALLESRVNLPQYPPTAIDYKCHLKKHNERQFFLELVKDISNDLDLTSLSYKILIFVCLMVDADRCSLFLVEGAAAGKKTLVSKFFDVHAGTPLLPCSSTENSNEVQVPWGKGIIGYVGEHGETVNIPDAYQDRRFNDEIDKLTGYKTKSLLCMPIRSSDGEIIGVAQAINKIPEGAPFTEDDEKVMQMYLPFCGIAISNAQLFAASRKEYERSRALLEVVNDLFEEQTDLEKIVKKIMHRAQTLLKCERCSVLLLEDIESPVVKFTKSFELMSPKCSADAENSFKESMEKSSYSDWLINNSIAELVASTGLPVNISDAYQDPRFDAEADQISGFHIRSVLCVPIWNSNHQIIGVAQVLNRLDGKPFDDADQRLFEAFVIFCGLGINNTIMYDQVKVEKQYQKDILQSLSDAVISTDMAGRIVTINDAALELLGCPLGDANHKSNKLLWEQNLIGRVVWEIVPIENLQMRLEDSLKSGAKHYVPEQSLIVGIYQLQMSESRVLHETQDYSILTVRDRINPDIFLPWNLPQTPQSQFITPEEVQILERSINLTVNPLTNPEGGVRGGLVVLEDISQEKRLKTTMYRYLTPHVAEQVMALGEDALMVGERKEVTVLFSDIRGYTTLTENLGAAEVVSLLNQYFETMVEAVFNYEGTLDKFIGDALMAVFGAPLPLTENHAWQAVQSALDMRQRLKEFNQRRIIQAQPQIKIGIGISSGEVVSGNIGSHKRMDYTVIGDGVNLSSRLETVTKEYGCDIILSEFTYQLCSDRIWVRQLDKIRVKGKHQAVNIYELISDRSTPLDDNTQEFLFHYHNGRTAYLVRDFTQAIACFNSAKHIRPTDQAVNIHLERAYNYQQTPPPPQWDGVWTIFTK

SEQ ID NO: 2 - DNA, 3198 bp, Artificial sequence
  CDS (1)..(3198): PDE11GAF/CyaB1-Chimera
atgggccatcatcatcatcatcatcatcatcatcacagcagcggccatatcgaaggtcgtcatagatccatggcagcctcccgcctggacttcggggaggtggaaactttcctggacaggcacccagagttgtttgaagattacttgatgcggaaggggaagcaggagatggttgaaaagtggctgcagaggcacagtcagggtcagggggctttaggtccaaggccctctttggctggtaccagcagcttggctcacagcacctgcagaggtggcagcagcgttggtggtggcactggaccaaatggctctgcccacagccagccccttcccggtggcggggactgtggtggggttcccttgagtcccagctgggccggtggcagcaggggcgatgggaacctgcagcggagagcttctcagaaagagctaaggaagagttttgcccgctccaaggccatccacgtgaacaggacctacgatgaacaggtgacctcccgggctcaggaacccctgagtagtgtgcgacggagggcacttctccggaaggcaagctccctgccccccaccacagcccatattctcagtgcgctgctggaatcgagagtgaatctgcctcagtatccccctacagccatcgactacaagtgccatctgaaaaagcataatgagcgtcagttctttctggaattggtcaaagatatctccaatgaccttgacctcaccagcctgagctacaagattctcatctttgtctgccttatggtggatgctgaccgctgctctcttttcctggtggaaggggcagctgctggcaagaagaccttggtctccaaattctttgatgtgcatgcaggaacccctctgctgccttgcagcagcacagagaactcaaatgaggtgcaggtcccctggggcaaaggtatcattggctatgtcggggagcatggagaaacggtcaacattcctgatgcctaccaggatcgacgattcaatgatgaaatcgacaagctaactggatacaagacaaaatcattattgtgcatgcctatccgaagcagtgatggtgagattattggtgtggcccaagcgataaataagattcctgaaggagctccatttactgaagatgatgaaaaagttatgcagatgtatcttccattttgtggaatcgccatatctaacgctcagctctttgctgcctcaaggaaagaatatgaaagaagcagagctttgctagaggtggttaatgacctctttgaagaacagactgacctggagaaaattgtcaagaaaataatgcatcgggcccaaactctgctgaaatgtgaacgctgttccgttttactcctagaggacatcgaatcaccagtggtgaaatttaccaaatcctttgaattgatgtccccaaagtgcagtgctgatgctgagaacagtttcaaagaaagcatggagaaatcatcatactccgactggctaataaataacagcattgctgagctggttgcttcaacaggccttccagtgaacatcagtgatgcctaccaggatccgcgctttgatgcagaggcagaccagatatctggttttcacataagatctgttctttgtgtccctatttggaatagcaaccaccaaataattggagtggctcaagtgttaaacagacttgatgggaaaccttttgatgatgcagatcaacgactttttgaggcttttgtcatcttttgtggacttggcatcaacaacacaattatgtatgatcaagtgaaggtcgagaaacaatatcaaaaagacattttacaaagcttgtcagatgctgtaatttctacagatatggccgggagaattgtcacaattaatgatgcagccttggaattactcggttgtcctttaggtgatgctaatcataaaagtaataagctgctgtgggaacaaaatttaattggtcgcgtagtttgggaaattgtaccaattgaaaatttgcagatgcgcttagaagatagtttaaaaagtggtgctaaacattatgtgccagaacaaagtttgatagtgggaatttatcaattacaaatgtctgaaagtcgggttttgcatgaaactcaagactactctattttgacagtacgcgatcgcatcaacccagatatttttctcccctggaatttaccccaaaccccccagtcgcaatttatcaccccggaagaagtacaaatcttagaacgcagtattaatcttaccgttaatcctttgacgaacccagaaggcggtgtccgtggtggtttggtagttttggaagatattagtcaagagaagcgcctcaaaactactatgtatcgctaccttacaccccatgtagctgaacaggtaatggctttaggggaagatgccttaatggttggtgaacgcaaggaggtgactgttttattttcagatatccgaggctacaccacacttacggaaaatctaggtgcggctgaagtggtatcactcctgaaccaatattttgaaacaatggttgaagcagttttcaactatgaaggcacactggataaatttatcggtgatgctttaatggctgtttttggtgcgccactaccactcacagaaaatcatgcttggcaagcagtacagtcagcattagatatgcgccaacgcctgaaggaatttaaccaacgacgcatcattcaggcacaaccacaaatcaaaatcggtattggtattagttctggagaagtagtttctggtaacatcggttctcacaagcgtatggattacacagtcattggtgatggtgtgaatttaagttcccgcttggaaactgtcaccaaagaatatggctgtgatattatcctcagtgagtttacttaccaattatgcagcgatcgcatttgggtacgtcagttagataaaatccgagtcaaagggaaacaccaagctgtcaatatctatgagttgattagcgatcgcagtactcccttagatgacaacacccaagagttcctctttcactatcataatggtcggactgcctacttagtccgcgattttacccaggcgatcgcttgttttaactcagctaaacatattcgacccacagaccaagctgtcaatattcacctagaacgcgcctacaattatcaacaaactccaccacctcctcaatgggacggcgtatggacaattttcacaaagtag

SEQ ID NO: 3 - protein, 1065 aa, Artificial sequence [sequence omitted]

SEQ ID NO: 4 - protein, 1065 aa, Artificial sequence [sequence omitted]

SEQ ID NO: 5 - DNA, 492 bp, Homo sapiens
  CDS (1)..(492): GAF-A PDE11
cttttcctggtggaaggggcagctgctggcaagaagaccttggtctccaaattctttgatgtgcatgcaggaacccctctgctgccttgcagcagcacagagaactcaaatgaggtgcaggtcccctggggcaaaggtatcattggctatgtcggggagcatggagaaacggtcaacattcctgatgcctaccaggatcgacgattcaatgatgaaatcgacaagctaactggatacaagacaaaatcattattgtgcatgcctatccgaagcagtgatggtgagattattggtgtggcccaagcgataaataagattcctgaaggagctccatttactgaagatgatgaaaaagttatgcagatgtatcttccattttgtggaatcgccatatctaacgctcagctctttgctgcctcaaggaaagaatatgaaagaagcagagctttgctagaggtggttaatgacctctttgaagaacagactgacctg

SEQ ID NO: 6 - protein, 164 aa, Homo sapiens
LFLVEGAAAGKKTLVSKFFDVHAGTPLLPCSSTENSNEVQVPWGKGIIGYVGEHGETVNIPDAYQDRRFNDEIDKLTGYKTKSLLCMPIRSSDGEIIGVAQAINKIPEGAPFTEDDEKVMQMYLPFCGIAISNAQLFAASRKEYERSRALLEVVNDLFEEQTDL

SEQ ID NO: 7 - DNA, 432 bp, Homo sapiens
  CDS (1)..(432): GAF-B PDE11
gttttactcctagaggacatcgaatcaccagtggtgaaatttaccaaatcctttgaattgatgtccccaaagtgcagtgctgatgctgagaacagtttcaaagaaagcatggagaaatcatcatactccgactggctaataaataacagcattgctgagctggttgcttcaacaggccttccagtgaacatcagtgatgcctaccaggatccgcgctttgatgcagaggcagaccagatatctggttttcacataagatctgttctttgtgtccctatttggaatagcaaccaccaaataattggagtggctcaagtgttaaacagacttgatgggaaaccttttgatgatgcagatcaacgactttttgaggcttttgtcatcttttgtggacttggcatcaacaacacaattatgtatgatcaagtgaag

SEQ ID NO: 8 - protein, 144 aa, Homo sapiens
VLLLEDIESPVVKFTKSFELMSPKCSADAENSFKESMEKSSYSDWLINNSIAELVASTGLPVNISDAYQDPRFDAEADQISGFHIRSVLCVPIWNSNHQIIGVAQVLNRLDGKPFDDADQRLFEAFVIFCGLGINNTIMYDQVK

SEQ ID NO: 9 - DNA, 1704 bp, Homo sapiens
  CDS (1)..(1704): GAF PDE11
atggcagcctcccgcctggacttcggggaggtggaaactttcctggacaggcacccagagttgtttgaagattacttgatgcggaaggggaagcaggagatggttgaaaagtggctgcagaggcacagtcagggtcagggggctttaggtccaaggccctctttggctggtaccagcagcttggctcacagcacctgcagaggtggcagcagcgttggtggtggcactggaccaaatggctctgcccacagccagccccttcccggtggcggggactgtggtggggttcccttgagtcccagctgggccggtggcagcaggggcgatgggaacctgcagcggagagcttctcagaaagagctaaggaagagttttgcccgctccaaggccatccacgtgaacaggacctacgatgaacaggtgacctcccgggctcaggaacccctgagtagtgtgcgacggagggcacttctccggaaggcaagctccctgccccccaccacagcccatattctcagtgcgctgctggaatcgagagtgaatctgcctcagtatccccctacagccatcgactacaagtgccatctgaaaaagcataatgagcgtcagttctttctggaattggtcaaagatatctccaatgaccttgacctcaccagcctgagctacaagattctcatctttgtctgccttatggtggatgctgaccgctgctctcttttcctggtggaaggggcagctgctggcaagaagaccttggtctccaaattctttgatgtgcatgcaggaacccctctgctgccttgcagcagcacagagaactcaaatgaggtgcaggtcccctggggcaaaggtatcattggctatgtcggggagcatggagaaacggtcaacattcctgatgcctaccaggatcgacgattcaatgatgaaatcgacaagctaactggatacaagacaaaatcattattgtgcatgcctatccgaagcagtgatggtgagattattggtgtggcccaagcgataaataagattcctgaaggagctccatttactgaagatgatgaaaaagttatgcagatgtatcttccattttgtggaatcgccatatctaacgctcagctctttgctgcctcaaggaaagaatatgaaagaagcagagctttgctagaggtggttaatgacctctttgaagaacagactgacctggagaaaattgtcaagaaaataatgcatcgggcccaaactctgctgaaatgtgaacgctgttccgttttactcctagaggacatcgaatcaccagtggtgaaatttaccaaatcctttgaattgatgtccccaaagtgcagtgctgatgctgagaacagtttcaaagaaagcatggagaaatcatcatactccgactggctaataaataacagcattgctgagctggttgcttcaacaggccttccagtgaacatcagtgatgcctaccaggatccgcgctttgatgcagaggcagaccagatatctggttttcacataagatctgttctttgtgtccctatttggaatagcaaccaccaaataattggagtggctcaagtgttaaacagacttgatgggaaaccttttgatgatgcagatcaacgactttttgaggcttttgtcatcttttgtggacttggcatcaacaacacaattatgtatgatcaagtgaag

SEQ ID NO: 10 - protein, 568 aa, Homo sapiens
MAASRLDFGEVETFLDRHPELFEDYLMRKGKQEMVEKWLQRHSQGQGALGPRPSLAGTSSLAHSTCRGGSSVGGGTGPNGSAHSQPLPGGGDCGGVPLSPSWAGGSRGDGNLQRRASQKELRKSFARSKAIHVNRTYDEQVTSRAQEPLSSVRRRALLRKASSLPPTTAHILSALLESRVNLPQYPPTAIDYKCHLKKHNERQFFLELVKDISNDLDLTSLSYKILIFVCLMVDADRCSLFLVEGAAAGKKTLVSKFFDVHAGTPLLPCSSTENSNEVQVPWGKGIIGYVGEHGETVNIPDAYQDRRFNDEIDKLTGYKTKSLLCMPIRSSDGEIIGVAQAINKIPEGAPFTEDDEKVMQMYLPFCGIAISNAQLFAASRKEYERSRALLEVVNDLFEEQTDLEKIVKKIMHRAQTLLKCERCSVLLLEDIESPVVKFTKSFELMSPKCSADAENSFKESMEKSSYSDWLINNSIAELVASTGLPVNISDAYQDPRFDAEADQISGFHIRSVLCVPIWNSNHQIIGVAQVLNRLDGKPFDDADQRLFEAFVIFCGLGINNTIMYDQVK

SEQ ID NO: 11 - DNA, 1425 bp, Anabaena PCC7120
  CDS (1)..(1425): Catalytic domain CyaB1 (V386)
gtcgagaaacaatatcaaaaagacattttacaaagcttgtcagatgctgtaatttctacagatatggccgggagaattgtcacaattaatgatgcagccttggaattactcggttgtcctttaggtgatgctaatcataaaagtaataagctgctgtgggaacaaaatttaattggtcgcgtagtttgggaaattgtaccaattgaaaatttgcagatgcgcttagaagatagtttaaaaagtggtgctaaacattatgtgccagaacaaagtttgatagtgggaatttatcaattacaaatgtctgaaagtcgggttttgcatgaaactcaagactactctattttgacagtacgcgatcgcatcaacccagatatttttctcccctggaatttaccccaaaccccccagtcgcaatttatcaccccggaagaagtacaaatcttagaacgcagtattaatcttaccgttaatcctttgacgaacccagaaggcggtgtccgtggtggtttggtagttttggaagatattagtcaagagaagcgcctcaaaactactatgtatcgctaccttacaccccatgtagctgaacaggtaatggctttaggggaagatgccttaatggttggtgaacgcaaggaggtgactgttttattttcagatatccgaggctacaccacacttacggaaaatctaggtgcggctgaagtggtatcactcctgaaccaatattttgaaacaatggttgaagcagttttcaactatgaaggcacactggataaatttatcggtgatgctttaatggctgtttttggtgcgccactaccactcacagaaaatcatgcttggcaagcagtacagtcagcattagatatgcgccaacgcctgaaggaatttaaccaacgacgcatcattcaggcacaaccacaaatcaaaatcggtattggtattagttctggagaagtagtttctggtaacatcggttctcacaagcgtatggattacacagtcattggtgatggtgtgaatttaagttcccgcttggaaactgtcaccaaagaatatggctgtgatattatcctcagtgagtttacttaccaattatgcagcgatcgcatttgggtacgtcagttagataaaatccgagtcaaagggaaacaccaagctgtcaatatctatgagttgattagcgatcgcagtactcccttagatgacaacacccaagagttcctctttcactatcataatggtcggactgcctacttagtccgcgattttacccaggcgatcgcttgttttaactcagctaaacatattcgacccacagaccaagctgtcaatattcacctagaacgcgcctacaattatcaacaaactccaccacctcctcaatgggacggcgtatggacaattttcacaaagtag

SEQ ID NO: 12 - protein, 474 aa, Anabaena PCC7120
  PEPTIDE (1)..(474): Catalytic domain CyaB1 (V386)
VEKQYQKDILQSLSDAVISTDMAGRIVTINDAALELLGCPLGDANHKSNKLLWEQNLIGRVVWEIVPIENLQMRLEDSLKSGAKHYVPEQSLIVGIYQLQMSESRVLHETQDYSILTVRDRINPDIFLPWNLPQTPQSQFITPEEVQILERSINLTVNPLTNPEGGVRGGLVVLEDISQEKRLKTTMYRYLTPHVAEQVMALGEDALMVGERKEVTVLFSDIRGYTTLTENLGAAEVVSLLNQYFETMVEAVFNYEGTLDKFIGDALMAVFGAPLPLTENHAWQAVQSALDMRQRLKEFNQRRIIQAQPQIKIGIGISSGEVVSGNIGSHKRMDYTVIGDGVNLSSRLETVTKEYGCDIILSEFTYQLCSDRIWVRQLDKIRVKGKHQAVNIYELISDRSTPLDDNTQEFLFHYHNGRTAYLVRDFTQAIACFNSAKHIRPTDQAVNIHLERAYNYQQTPPPPQWDGVWTIFTK

SEQ ID NO: 13 - protein, 1064 aa, Artificial sequence
  PEPTIDE (1)..(1064): PDE11GAF/CyaB1-Chimera
MGHHHHHHHHHHSSGHIEGRHRSMAASRLDFGEVETFLDRHPELFEDYLMRKGKQEMVEKWLQRHSQGQGALGPRPSLAGTSSLAHSTCRGGSSVGGGTGPNGSAHSQPLPGGGDCGGVPLSPSWAGGSRGDGNLQRRASQKELRKSFARSKAIHVNRTYDEQVTSRAQEPLSSVRRRALLRKASSLPPTTAHILSALLESRVNLPQYPPTAIDYKCHLKKHNERQFFLELVKDISNDLDLTSLSYKILIFVCLMVDADRCSLFLVEGAAAGKKTLVSKFFDVHAGTPLLPCSSTENSNEVQVPWGKGIIGYVGEHGETVNIPDAYQDRRFNDEIDKLTGYKTKSLLCMPIRSSDGEIIGVAQAINKIPEGAPFTEDDEKVMQMYLPFCGIAISNAQLFAASRKEYERSRALLEVVNDLFEEQTDLEKIVKKIMHRAQTLLKCERCSVLLLEDIESPVVKFTKSFELMSPKCSADAENSFKESMEKSSYSDWLINNSIAELVASTGLPVNISDAYQDPRFDAEADQISGFHIRSVLCVPIWNSNHQIIGVAQVLNRLDGKPFDDADQRLFEAFVIFCGLGINNTIMYDQVKVEKQYQKDILQSLSDAVISTDMAGRIVTINDAALELLGCPLGDANHKSNKLLWEQNLIGRVVWEIVPIENLQMRLEDSLKSGAKHYVPEQSLIVGIYQLQMSESRVLHETQDYSILTVRDRINPDIFLPWNLPQTPQSQFITPEEVQILERSINLTVNPLTNPEGGVRGGLVVLEDISQEKRLKTTMYRYLTPHVAEQVMALGEDALMVGERKEVTVLFSDIRGYTTLTENLGAAEVVSLLNQYFETMVEAVFNYEGTLDKFIGDALMAVFGAPLPLTENHAWQAVQSALDMRQRLKEFNQRRIIQAQPQIKIGIGISSGEVVSGNIGSHKRMDYTVIGDGVNLSSRLETVTKEYGCDIILSEFTYQLCSDRIWVRQLDKIRVKGKHQAVNIYELISDRSTPLDDNTQEFLFHYHNGRTAYLVRDFTQIACFNSAKHIRPTDQAVNIHLERAYNYQQTPPPPQWDGVWTIFTK

SEQ ID NO: 14 - DNA, 489 bp, Homo sapiens
  CDS (1)..(489): GAF-A PDE11
ttcctggtggaaggggcagctgctggcaagaagaccttggtctccaaattctttgatgtgcatgcaggaacccctctgctgccttgcagcagcacagagaactcaaatgaggtgcaggtcccctggggcaaaggtatcattggctatgtcggggagcatggagaaacggtcaacattcctgatgcctaccaggatcgacgattcaatgatgaaatcgacaagctaactggatacaagacaaaatcattattgtgcatgcctatccgaagcagtgatggtgagattattggtgtggcccaagcgataaataagattcctgaaggagctccatttactgaagatgatgaaaaagttatgcagatgtatcttccattttgtggaatcgccatatctaacgctcagctctttgctgcctcaaggaaagaatatgaaagaagcagagctttgctagaggtggttaatgacctctttgaagaacagactgacctg

SEQ ID NO: 15 - protein, 163 aa, Homo sapiens
FLVEGAAAGKKTLVSKFFDVHAGTPLLPCSSTENSNEVQVPWGKGIIGYVGEHGETVNIPDAYQDRRFNDEIDKLTGYKTKSLLCMPIRSSDGEIIGVAQAINKIPEGAPFTEDDEKVMQMYLPFCGIAISNAQLFAASRKEYERSRALLEVVNDLFEEQTDL

(19) United States
(12) Patent Application Publication
(10) Pub. No.: US 2009/0298108 A1
(43) Pub. Date: Dec. 3, 2009

(54) Method for Identifying PDE11 Modulators

(76) Inventors: Joachim Schultz, Entringen (DE); Marco Gross-Langenhoff, Tubingen (DE)

Correspondence Address: NATH & ASSOCIATES PLLC, 112 South West Street, Alexandria, VA, 22314, US

(21) Appl. No.: 11/884,766

(22) PCT Filed: May 18, 2005
(86) PCT No.: PCT/EP05/52273

§ 371 Date: Sep. 26, 2007

(30) Foreign Application Priority Data
Feb. 28, 2005 (DE) 10 2005 009 672.7
Mar. 1, 2005 (DE) 10 2005 009 848.7

(51) Int. Cl. C12Q 1/44; C12N 9/16; C12N 15/11; C12N 15/00; C12N 5/06; C12N 1/21; C12P 21/02

(52) U.S. Cl. 435/19; 435/196; 536/23.2; 435/320.1; 435/325; 435/252.3; 435/69.7

(57) ABSTRACT

The invention relates to a novel GAF_{A} domain-containing polypeptide, to the GAF_{A} domain of a human phosphodiesterase 11 (PDE11) and to the adenylate cyclase catalytic domain. The use of said polypeptide in a method for identifying PDE-11 modulators is also disclosed.

### TECHNICAL FIELD

The present invention concerns a novel polypeptide containing the GAF_{A} domain and GAF_{B} domain of a human phosphodiesterase 11 (PDE11) and the catalytic domain of an adenylate cyclase, as well as use of this polypeptide in a method for identification of PDE11-modulators.

### PRIOR ART

Phosphodiesterases (=PDEs) are eukaryotic proteins and are known as modulators of the cyclic nucleotides cAMP and cGMP. PDEs are divided into three classes (I, II, and III), of which only Class I, with its 11 PDE families (referred to as PDE1 through -11), occurs in mammals.

GAF domains are ubiquitous in all areas of life and were defined by Aravind and Ponting based on protein structure and sequence comparisons (Aravind L. and Poting C. P.: The GAF domain: An evolutionary link between diverse phototransducing proteins, 1997, TIBS, 22, 458-459). PDE2, PDE5, and PDE6 contain so-called cGMP-binding GAF domains, which play a role in allosteric activation of PDEs.

Various isoforms of human PDE11 have been cloned and characterized (Hetman et al., PNAS 2000, 97, 12891 to 12895 and Soderling et al., Current Opinion in Cell Biology 2000, 12, 174-179).

Adenylate cyclases (=ACs) catalyze the conversion of ATP into cAMP in all areas of life (Cooper D. M.: Regulation and organization of adenylyl cyclases and cAMP. 2003, Biochem J., 375 (Pt. 3), 517-29; Tang W. J. and Gilman A. G.: Construction of a soluble adenylyl cyclase activated by Gsα and forskolin. 1995, Science, 268, 1769-1772). Based on sequence comparisons and structural considerations, they are divided into five Classes (I through V). The bacterial Class III ACs from Cyanobacteria, particularly from Nostoc sp. PCC 7120, to which CyaB1 also belongs, are of molecular biological interest. The Cyanobacteria Acs CyaB1 and CyaB2 also contain N-terminal GAF domains that are structurally similar to those of the PDEs, but have cAMP as an activating ligand. The nine known families of Class III Acs in humans are all membrane-bound and are regulated via G-proteins (Tang W. J. and Gilman A. G.: Construction of a soluble adenylyl cyclase activated by Gsα and forskolin. 1995, Science, 268, 1769-1772). A combination with GAF domains is not known in the art.

The construction of a chimera from the GAF domains of rat PDE2 and the catalytic centre of adenylate cyclase CyaB1 has already been described (Kanacher T., Schultz A., Linder J. U., and Schultz J. E.: A GAF domain-regulated adenylyl cyclase from Anabaena is a self-activated cAMP switch. 2002, EMBO J., 21, 3672-3680).

A chimera of human PDE11 and bacterial adenylate cyclase is not known in the art. Moreover, the use of such a chimera in a method for the identification of PDE11-modulators is also not known in prior art.

### DESCRIPTION OF THE INVENTION

The purpose of the invention is to provide a process for the identification of PDE11-modulators.

This objective is achieved by providing the polypeptide according to the invention, comprising, functionally linked, (a) the GAF_{A} domain and GAF_{B} domain of a human phosphodiesterase 11 (PDE11) or its functionally equivalent variants and (b) the catalytic domains of an adenylate cyclase or its functionally equivalent variants, and its use in a process for the identification of PDE11-modulators.

Surprisingly, it was found that a chimeric protein composed of N-terminal human PDE11-GAF domains and a C-terminal catalytic centre of an adenylate cyclase is suitable as an effector molecule. In chimeric proteins, the GAF domains are the activation domains that modify their conformation during ligand formation and thus modulate the catalytic activity of the adenylate cyclase domain, which serves as a read-out.

Furthermore, surprisingly, it was found that cGMP selectively activates the GAF domain of PDE11 as agonist.

These results were particularly surprising since, for example, the GAF domain of PDE11A4 shows only 26% identity to the GAF domain of CyaB1 and a functional activating ligand of the GAF domain of PDE11A4 has until now been unknown (Yuasa K., Kanoh Y., Okumura K., Omori K. Genomic organization of the human phosphodiesterase PDE11A gene. Evolutionary relatedness with other PDEs containing GAF domains. Eur J Biochem. 2001, 268, 168-78).

The present invention makes it possible to identify PDE11-modulators, i.e., PDE11-antagonists or PDE11 agonists, which act not via binding and blocking of the catalytic centre of the PDE11, but via allosteric regulation on the N-terminal of the PDE11, i.e., on the GAF domain.

As mentioned above, the invention concerns a polypeptide comprising, functionally linked, (a) the GAF_{A} domain and GAF_{B} domain of a human phosphodiesterase 11 (PDE11) or its functionally equivalent variants and (b) the catalytic domain of an adenylate cyclase or its functionally equivalent variants.

The term human phosphodiesterase, or PDE, denotes an enzyme of human origin that is capable of converting cAMP or cGMP into the corresponding inactivated 5′ monophosphate. Based on their structure and properties, the PDEs are classified into various families. A human phosphodiesterase 11, also referred to as PDE11, particularly denotes an enzyme family of human origin that is capable of converting cGMP into the inactive 5′ monophosphate.

PDE11s suitable for use in the invention include all PDE11s that have a GAF_{A} domain and a GAF_{B} domain. The GAF domains of PDE11 are located in the protein as a tandem N-terminal. The GAF domain closest to the N-terminal is referred to as GAF_{A}, and the immediately following domain is referred to as GAF_{B}. The beginning and end of the GAF domains can be determined by means of protein sequence comparisons. A SMART sequence comparison (Schultz J., Milpetz F., Bork P., and Poting C. P.: SMART a simple modular architecture research tool: Identification of signaling domains. 1998, PNAS, 95, 5857-5864), for example, yields the isoform PDE11A4: L240 to L403 (SEQ. I.D. NO. 6) for GAF_{A} and V425 to K591 (SEQ. I.D. NO. 8) for GAF_{B}.

The term adenylate cyclase refers to an enzyme that is capable of converting ATP into cAMP. Accordingly, adenylate cyclase activity refers the amount of ATP converted or the amount of cAMP formed by the polypeptide according to the invention in a particular period of time.

A catalytic domain of an adenylate cyclase refers to a portion of the amino acid sequence of an adenylate cyclase that is necessary for the adenylate cyclase to display its property of converting ATP into cAMP, i.e. is still essentially functional and thus shows adenylate cyclase activity.

Iterative shortening of the amino acid sequence and subsequent measurement of adenylate cyclase activity makes it possible to easily determine the catalytic domains of an adenylate cyclase.

For example, the determination of adenylate cyclase activity may take place through measurement of the conversion of radioactive [α-³²P]-ATP into [))000 -³²P]-cAMP.

Generally speaking, adenylate cyclase activity can easily be determined by measuring the resulting cAMP or antibody formation. For this purpose, there are various commercial assay kits such as the cAMP [³H-] or [¹²⁵-I] BioTrak® cAMP SPA-Assay from Amersham® or the AlphaScreen® or Lance® cAMP Assay from PerkinElmer®: these are all based on the principle that during the AC reaction, unlabeled cAMP originates from ATP. This competes with exogenously added 3H-, 125I-, or Biotin-labeled cAMP for binding to a cAMP-specific antibody. In the non-radioactive Lance® Assay, Alexa®-Flour is bound to the antibody, which, with the tracer, generates a TR-FRET signal at 665 nm. The more unlabeled cAMP is bound, the weaker the signal generated by the labeled cAMP. A standard curve can be used in order to classify the signal strength of the corresponding cAMP concentration.

Analogously to the High-Efficiency Fluorescence Polarization (HEFP™)-PDE Assay from Molecular Devices, which is based on IMAP technology, one can use fluorescently, rather than radioactively labeled substrate. In the HEFP-PDE Assay, fluorescein-labeled cAMP (Fl-cAMP) is used, which is converted by the PDE into fluorescein-labeled 5′ AMP (Fl-AMP). The Fl-AMP selectively binds to special beads, thus causing the fluorescence to be strongly polarized. Fl-cAMP does not bind to the beads, so an increase in polarization is proportional to the amount of Fl-AMP generated. For a corresponding AC-test, fluorescence-labeled ATP may be used instead of Fl-cAMP, and beads that selectively bind to Fl-cAMP instead of Fl-cAMP (e.g. beads that are loaded with cAMP antibodies) may be used.

“Functionally equivalent variants” of polypeptides or domains, i.e., sequence segments of polypeptides with a particular function, refers to polypeptides and/or domains that differ structurally as described below but still fulfill the same function. Functionally equivalent variants of domains can be easily found by a person skilled in the art, as described below in further detail, by variation and functional testing of the corresponding domains, by sequence comparisons with corresponding domains of other known proteins, or by hybridization of the corresponding nucleic acid sequences coding for these domains with suitable sequences from other organisms.

“Functional linkage” refers to linkages, preferably covalent bonds of domains that lead to an arrangement of the domains so that they can fulfill their function. For example, functional binding of the GAF_{A} domain, GAF_{B} domain, and the catalytic domain of adenylate cyclase refers to binding of these domains that leads to arrangement of the domains so that the GAF domains change their conformation due to ligand binding, for example by cGMP or PDE11 modulators and thus modulate the catalytic activity of the adenylate cyclase domain. Moreover, for example, a functional binding of the GAF_{A} domain and the GAF_{B} domain refers to binding of these domains that leads to ordering of the domains in such a way that the GAF_{A} domain and the GAF_{B} domain change their conformation together as GAF domains in ligand binding, for example by cGMP or PDE11 modulators.

Preferably, the human phosphodiesterases 11 (PDE11) that can be used for the GAF domains, GAF_{A} and GAF_{B,} are selected from the group of the isoforms PDE11A (Accession: NP_{—}058649/BAB16371), PDE11A1 (Accession: BAB62714/CAB82573), PDE11A2 (Accession: BAB16372), PDE11A3 (Accession: BAB62713) and PDE11A4 (Accession: BAB62712) or their respective functionally equivalent variants, and use according to the invention of the GAF domains of the isoform PDE 11A4 or its functional equivalent variants is particularly preferred.

In a preferred embodiment, the GAF_{A} domain of the polypeptide according to the invention shows an amino acid sequence containing the amino acid sequence having SEQ. I.D. NO. 6 or a sequence derived from this sequence by substitution, insertion, or deletion of amino acids, that has an identity of at least 90%, preferably at least 91%, more preferably at least 92%, more preferably at least 93%, more preferably at least 94%, more preferably at least 95%, more preferably at least 96%, more preferably at least 97%, more preferably at least 98%, more preferably at least 99% at the amino acid level with the sequence having SEQ. I.D. NO. 6 and the property of a GAF_{A} domain.

Instead of SEQ. I.D. NO. 6, SEQ ID NO. 15 may be used analogously for the entire description. In SEQ. I.D. NO. 15, the N-terminus of the GAF_{A} domain is shortened by one amino acid (L240) with respect to SEQ. I.D. NO. 6.

In this case, this may be a natural functional equivalent variant of the GAF_{A} domain that, as described above, can be found through identity comparison of the sequences with other proteins or an artificial GAF_{A} domain that has been converted based on the sequence having SEQ. I.D. NO. 6 by artificial variation, for example through substitution, insertion, or deletion of amino acids.

The term “substitution” refers in the description to the substitution of one or several amino acids by one or several amino acids. Preferably, so-called conservative exchanges are to be carried out, in which the replaced amino acid has a property similar to that of the original amino acid, for example replacement of Glu by Asp, Gln by Asn, Val by Ile, Leu by Ile, or Ser by Thr.

Deletion is the replacement of an amino acid through direct bonding. Preferred positions for deletion are the terminals of the polypeptide and the links between the individual protein domains.

Insertions are inclusions of amino acids in the polypeptide chain, in which a direct bond is formally replaced by one or more amino acids.

Identity between two proteins refers to the identity of the amino acids over the entire respective protein link, specifically the identity that is calculated by comparison using Lasergene Software of DNASTAR, Inc., Madison, Wis. (USA) using the Clustal Method (Higgins D. G. Sharp P. M.: Fast and sensitive multiple sequence alignments on a microcomputer. Comput Appl. Biosci. 1989 April; 5 (2): 151-1), setting the following perimeters:

Multiple Alignment Perimeter:

Gap penalty 10

Gap length penalty 10

Pairwise Alignment Perimeter:

K-tuple 1

Gap penalty 3

Window 5

Diagonals saved 5

A protein or a domain having an identity of at least 90% at the amino acid level with the sequence SEQ. I.D. NO. 6 will thus denote a protein and/or a domain which, after comparison of its sequence to the sequence SEQ. I.D. NO. 6, particularly according to the above program logarithm with the above perimeter set, shows an identity of at least 90%.

The property of a GAF_{A} domain specifically refers to its function of binding cGMP, in particular together with the GAF_{B} domain.

In a further preferred embodiment, the GAF_{A} domain of the polypeptide according to the invention shows the amino acid sequence having SEQ. I.D. NO. 6.

In a preferred embodiment, the GAF_{B} domain of the polypeptide according to the invention shows an amino acid sequence containing the amino acid sequence having SEQ. I.D. NO. 8 or a sequence derived from this sequence by substitution, insertion, or deletion of amino acids, that has an identity of at least 90%, preferably at least 91%, more preferably at least 92%, more preferably at least 93%, more preferably at least 94%, more preferably at least 95%, more preferably at least 96%, more preferably at least 97%, more preferably at least 98%, more preferably at least 99% of the amino acid level with the sequence SEQ. I.D. NO. 8 and the property of a GAF_{B} domain.

In this case, it may be a natural functional equivalent variant of the GAF_{B} domain which, as described above, can be found through identity comparison of the sequences with other proteins, or an artificial GAF_{B} domain which was converted based on the sequence having SEQ. I.D. NO. 6 by artificial variation, for example through substitution, insertion, or deletion of amino acids as described above.

Specifically, the property of a GAF_{B} domain denotes its function of being responsible for dimer formation, and specifically its function, together with the GAF_{A} domain, via binding of the cGMP of PDE11 to activate, or through binding of PDE11 modulators, to modulate the PDE11 activity, i.e., to increase or lower it.

In a further embodiment, the GAF_{B} domain of the polypeptide according to the invention has amino acid sequence SEQ. I.D. NO. 8.

In a further preferred embodiment of the polypeptide according to the invention, the functionally linked GAF_{A} domain and GAF_{B} domain, i.e., the complete GAF domain, show a human phosphodiesterase 11 (PDE11) or its functionally equivalent variants of an amino acid sequence, containing the amino acid sequence SEQ. I.D. NO. 10 or a sequence derived from this sequence by substitution, insertion, or deletion of amino acids, which shows an identity of at least 70%, preferably at least 75%, more preferably at least 80%, more preferably at least 85%, more preferably at least 90%, more preferably at least 93%, more preferably at least 95%, more preferably at least 97%, more preferably at least 98%, more preferably at least 99% at the amino acid level with sequence SEQ. I.D. NO. 10 and the regulatory property of the GAF domain of a human phosphodiesterase 11 (PDE11), with the amino acid sequences of the GAF_{A} domain acquired, SEQ. I.D. NO. 6 and the GAF_{B} domain, SEQ. I.D. NO. 8 varying through substitution, insertion, or deletion of amino acids by a maximum amount of 10%, more preferably a maximum of 9%, more preferably a maximum of 8%, more preferably a maximum of 7%, more preferably a maximum of 6%, more preferably a maximum of 5%, more preferably a maximum of 4%, more preferably a maximum of 3%, more preferably a maximum of 2%, more preferably a maximum of 1%, and more preferably a maximum of 0.5%.

In particular, the N-terminal residue of the particularly preferred GAF domain SEQ. I.D. NO. 10 is freely variable from the N-terminal to the GAF_{A} domain SEQ. ID. NO. 6, and in particular, can be shortened. Preferably, the N-terminal residue of the particularly preferred GAF domain SEQ. I.D. NO. 10 should be capable of shortening by 100 amino acid, more preferably by 90 amino acids, more preferably by 80 amino acids, more preferably by 70 amino acids, more preferably by 60 amino acids, more preferably by 50 amino acids, more preferably by 40 amino acids, more preferably by 30 amino acids, more preferably by 20 amino acids, more preferably by 10 amino acids, and more preferably by 5 amino acid N-terminals.

The amino acid partial sequences of the GAF_{A} domain SEQ. I.D. NO. 6 and the GAF_{B} domain SEQ. I.D. NO. 8 can be varied by substitution, insertion, or deletion of amino acids by a maximum of 10%, preferably a maximum of 9%, preferably a maximum of 8%, preferably a maximum of 7%, preferably a maximum of 6%, preferably a maximum of 5%, preferably a maximum of 4%, preferably a maximum of 3%, preferably a maximum of 2%, preferably a maximum of 1%, and preferably a maximum of 0.5% without this causing a loss of the respective above-described functions.

Preferably, the functionally linked GAF_{A} domain and GAF_{B} domain, i.e., the complete GAF domain, shows a human phosphodiesterase 11 (PDE11) or its functionally equivalent variants of an amino acid sequence selected from the group

(a) N-terminus of human PDE11A4 of amino acid M24 up to amino acid K591 or

(b) SEQ. I.D. NO. 10.

For the portion of the catalytic domain of an adenylate cyclase of the polypeptide according to the invention, adenylate cyclases are preferably used that in natural form show a GAF domain. Especially preferred adenylate cyclases are adenylate cyclases of bacterial origin, particularly from Cyanobacteria, which show a GAF domain in natural form or their respective functionally equivalent variants.

Particularly preferred adenylate cyclases are selected from the group:

(a) Adenylate cyclase from Anabaena sp. PCC 7120 or their functionally equivalent variants,

(b) Adenylate cyclase from Anabaena variabili ATTC 29413 or its functionally equivalent variants,

(c) Adenylate cyclase from Nostoc punctiforme PCC 73102 or its functionally equivalent variants,

(d) Adenylate cyclase from Trichodesmium erythraeum IMS 101 or its functionally equivalent variants,

(e) Adenylate cyclase from Bdellovibrio bacteriovorus HD 100 or its functionally equivalent variants,

(f) Adenylate cyclase from Magnetococcus sp. MC-1 or its functionally equivalent variants.

Particularly preferred adenylate cyclases are adenylate cyclases from Anabaena sp. PCC 7120 of the isoform CyaB1 or CyaB2, particularly CyaB1 (Accession: NP_{—}486306, D89623) or their functionally equivalent variants.

In a preferred embodiment, the catalytic domain of an adenylate cyclase or its functionally equivalent variants show an amino acid sequence containing the amino acid sequence SEQ. I.D. NO. 12 or a sequence derived from this sequence by substitution, insertion, or deletion of amino acids, which has an identity of at least 90%, preferably at least 91%, more preferably at least 92%, more preferably at least 93%, more preferably at least 94%, more preferably at least 95%, more preferably at least 96%, more preferably at least 97%, more preferably at least 98%, more preferably at least 99% at the amino acid level with the sequence SEQ. I.D. NO. 12 and the catalytic property of an adenylate cyclase.

In this case, it may be a natural functional equivalent variant of the catalytic domain of an adenylate cyclase which, as described above, can be found through identity comparison of the sequences with other adenylate cyclases or an artificial catalytic domain of an adenylate cyclase which was converted based on the sequence SEQ. I.D. NO. 12 by artificial variation, for example by substitution, insertion, or deletion of amino acids, as described above.

The property of a catalytic domain of an adenylate cyclase denotes the above described catalytic property of an adenylate cyclase, particularly the capacity to convert ATP into cAMP.

Preferably, the catalytic domain of an adenylate cyclase or its functionally equivalent variant shows an amino acid sequence selected from the group:

(a) C-terminal of CyaB1 of the amino acid L386 through K859, with L386 being of CyaB1 being replaced by V386 or

(b) SEQ. I.D. NO. 12.

In a particularly preferred embodiment, the polypeptide according to the invention includes the amino acid sequence SEQ. I.D. NO. 1 or SEQ. I.D. NO. 4 or a sequence derived from these sequences by substitution, insertion, or deletion of amino acids, that has an identity of at least 70%, preferably at least 75%, more preferably at least 80%, more preferably at least 85%, more preferably at least 90%, more preferably at least 93%, more preferably at least 95%, more preferably at least 97%, more preferably at least 98%, more preferably at least 99% on an amino acid level with the sequence SEQ. I.D. NO. 1 or 4 and the regulatory properties of the GAF domain of a human phosphodiesterase 11 (PDE11) and the catalytic properties of an adenylate cyclase, with the obtained amino acid sequences of the GAF_{A} domain, SEQ. I.D. NO. 6, the GAF_{B} domain, SEQ. I.D. NO. 8, and the catalytic domain of adenylate cyclase, SEQ. I.D. NO. 12, varying by a maximum of 10% through substitution, insertion, or deletion of amino acids.

Instead of SEQ. I.D. NO. 4, SEQ. I.D. NO. 13 may be used analogously for the entire description. In SEQ ID NO. 13 the amino acid A1020 is missing in comparison to SEQ. I.D. NO. 4.

In particular, the N-terminal residue of the particularly preferred polypeptide according to the invention SEQ. I.D. NO. 1 and SEQ. I.D. NO. 4 is freely variable, and particularly capable of shortening from the N-terminal to the GAF_{A} domain SEQ. I.D. NO. 6. Preferably, the N-terminal residue of the particularly preferred polypeptide according to the invention SEQ. I.D. NO. 1 or SEQ. I.D. NO. 4 can be shortened by 100 amino acids, more preferably by 90 amino acids, more preferably by 80 amino acids, more preferably by 70 amino acids, more preferably by 60 amino acids, more preferably by 50 amino acids, more preferably by 40 amino acids, more preferably by 30 amino acids; more preferably by 20 amino acids, more preferably by 10 amino acids, and more preferably by 5 amino acid N-terminals.

The amino acid partial sequences of GAF_{A} domain SEQ. I.D. NO. 6, GAF_{B} domain SEQ. ID. NO. 8, and the catalytic domains of adenylate cyclase, SEQ. I.D. NO. 12, can be varied by substitution, insertion, or deletion of amino acids by a maximum of 10%, more preferably a maximum of 9%, more preferably a maximum of 8%, more preferably a maximum of 7%, more preferably a maximum of 6%, more preferably a maximum of 5%, more preferably a maximum of 4%, more preferably a maximum of 3%, more preferably a maximum of 2%, more preferably a maximum of 1%, more preferably a maximum of 0.5% without this causing a loss of the respective above described function.

In a particularly preferred embodiment, the chimeric polypeptide N-terminal from M24 up to K591 according to the invention contains the N-terminal of human PDE11A4 (Accession: BAB62712). To this is attached the C-terminal of V386 that was mutated from L386 on insertion of the cloning interface up to K859 of the C-terminal of CyaB1 (Accession: NP_{—}486306).

Particularly preferred is a polypeptide according to the invention including the amino acid sequence having SEQ. I.D. NO. 1 or SEQ. I.D. NO. 4.

Even more particularly preferred polypeptides according to the invention are polypeptides with the amino acid sequence having SEQ. I.D. NO. 1 or SEQ. I.D. NO. 4.

In a further embodiment, the invention also concerns polynucleotides, also referred to in the following as nucleic acids, coding for one of the above-described polypeptides according to the invention.

All of the polynucleotides or nucleic acids mentioned in the description may, for example, be an RNA, DNA, or cDNA sequence.

Particularly preferred polynucleotides according to the invention contain as partial sequences

(a) SEQ. I.D. NO. 5 or a nucleic acid sequence that hybridizes with the nucleic acid sequence having SEQ. I.D. NO. 5 under stringent conditions and

(b) SEQ. I.D. NO. 7 or a nucleic acid sequence that hybridizes with the nucleic acid sequence having SEQ. I.D. NO. 7 under stringent conditions and

(c) SEQ. I.D. NO. 11 or a nucleic acid sequence that hybridizes with the nucleic acid sequence having SEQ. I.D. NO. 11 under stringent conditions.

SEQ. I.D. NO. 5 constitutes a particularly preferred partial nucleic acid sequence coding for the particularly preferred GAF_{A} domain SEQ. I.D. NO. 6.

SEQ. I.D. NO. 7 constitutes a particularly preferred partial nucleic acid sequence coding for the particularly preferred GAF_{B} domain SEQ. I.D. NO. 8.

SEQ. I.D. NO. 11 constitutes a particularly preferred partial nucleic acid sequence coding for the particularly preferred catalytic domain of an adenylate cyclase having SEQ. I.D. NO. 12.

Further natural examples of nucleic acids and/or partial nucleic acids coding for the above described domains can also be easily found by a method known in the art based on the above described partial nucleic acid sequences, particularly based on the sequences having SEQ. I.D. NO. 5, 7, or 11 from various organisms whose genomic sequence is not known, by means of hybridization techniques.

Hybridization may take place under moderate (low stringency) or preferably under stringent (high stringency) conditions.

Examples of such hybridization conditions are described in Sambrook, J., Fritsch, E. F., Maniatis, T., in: Molecular Cloning (A Laboratory Manual), 2nd Edition, Cold Spring Harbor Laboratory Press, 1989, pp. 9.31-9.57 or in Current Protocols in Molecular Biology, John Wiley & Sons, N.Y. (1989), 6.3.1-6.3.6.

For example, the conditions may be selected during the washing step from the area of conditions limited by those with low stringency (with 2×SSC at 50° C.) and those with high stringency (with 0.2×SSC at 50° C., preferably at 65° C.) (20×SSC: 0.3 M sodium citrate, 3 M sodium chloride, pH 7.0).

In addition, the temperature during the washing step may be increased from moderate conditions at room temperature, 22° C., to stringent conditions at 65° C.

Both perimeters, salt concentration and temperature, may be simultaneously varied, or one of the two perimeters may be kept constant and only the other varied. During hybridization, denatured agents such as formamide or SDS may also be used. In the presence of 50% formamide, hybridization is preferably carried out at 42° C.

A few examples of conditions for hybridization in the washing step are given below:

(1) Hybridization Conditions with e.g.

(i) 4×SSC at 65° C., or

(ii) 6×SSC at 45° C., or

(iii) 6×SSC at 68° C., 100 mg/mL denatured fish sperm DNA, or

(iv) 6×SSC, 0.5% SDS, 100 mg/mL denatured, fragmented salmon sperm DNA at 68° C., or

(v) 6×SSC, 0.5% SDS, 100 mg/mL denatured, fragmented salmon sperm DNA, 50% formamide at 42° C., or

(vi) 50% formamide 4×SSC at 42° C., or

(vii) 50% (vol/vol) formamide 0.1% bovine serum albumin, 0.1% Ficoll, 0.1% polyvinylpyrrolidone, 50 mM sodium phosphate buffer, pH 6.5, 750 mM NaCl, 75 mM sodium citrate at 42° C., or

(viii) 2× or 4×SSC at 50° C. (moderate conditions), or

(ix) 30 to 40% formamide, 2× or 4×SSC at 42° C. (moderate conditions).

(2) Wash Steps for 10 Minutes Each with e.g.

(i) 0.015 M NaCl/0.0015 M sodium citrate/0.1% SDS at 50° C., or

(ii) 0.1×SSC at 6520 C., or

(iii) 0.1×SSC, 0.5% SDS at 68° C., or

(iv) 0.1×SSC, 0.5% SDS, 50% formamide at 42° C., or

(v) 0.2×SSC, 0.1% SDS at 42° C., or

(vi) 2×SSC at 65° C. (moderate conditions).

A particularly preferred polynucleotide according to the invention coding for a polypeptide according to the invention contains the nucleic acid sequence SEQ. I.D. NO. 2.

An even more preferable polynucleotide according to the invention coding for a polypeptide according to the invention shows the nucleic acid sequence SEQ. I.D. NO. 2.

The polypeptide according to the invention can preferably be manufactured in that an above-described polynucleotide coding for a polypeptide according to the invention is cloned in a suitable expression vector, a host cell is transformed with this expression vector, this host cell is expressed under expression of the polypeptide according to the invention, and the protein according to the invention is then isolated.

The invention therefore concerns a process for the manufacture of a polypeptide according to the invention through cultivation of a recombinant host cell, expression, and isolation of the polypeptide according to the invention.

The transformation methods are known to a person skilled in the art, and these are described e.g., in Sambrook, J., Fritsch, E. F., Maniatis, T., in: Molecular Cloning (A Laboratory Manual), 2nd Edition, Cold Spring Harbor Laboratory Press, 1989, pp. 9.31-9.57.

The invention also concerns a recombinant plasmid vector, specifically an expression vector comprising a polynucleotide according to the invention coding for a polypeptide according to the invention.

The type of the expression vector is not critical. Any expression vector may be used that is capable of expressing the desired polypeptide in a corresponding host cell. Suitable expression systems are known to a person skilled in the art.

Preferred expression vectors are pQE30 (Quiagen), PQE60 (Quiagen), pMAL (NEB), pIRES, PIVEX2.4a (ROCHE), PIVEX2.4b (ROCHE), PIVEX2.4c (ROCHE), pUMVC1 (Aldevron), pUMVC2 (Aldevron), pUMVC3 (Aldevron), pUMVC4a (Aldevron), pUMVC4b (Aldevron), pUMVC7 (Aldevron), pUMVC6a (Aldevron), pSP64T, pSP64TS, pT7TS, pCro7 (Takara), pKJE7 (Takara), pKM260, pYes260, pGEM-Teasy.

The invention also concerns a recombinant host cell comprising a plasmid vector according to the invention. This transformed host cell is preferably capable of expressing the polypeptide according to the invention.

The type of host cell is not critical. Both prokaryotic host cells and eukaryotic host cells are suitable. Any host cell may be used that is capable with a corresponding expression vector of expressing the desired polypeptide. Suitable expression systems composed of expression vectors and host cells are known to a person skilled in the art.

Examples of preferred host cells include prokaryotic cells such as E. coli, Corynebacteria, yeasts, Streptomycetes, or eukaryotic cells such as CHO, HEK293, or insect cell lines such as SF9, SF21, Xenopus Oozytes.

The cultivation conditions of the transformed host cells, such as culture medium composition and fermentation conditions are known to a person skilled in the art and depend on the host cell selected.

The isolation and purification of the polypeptide may take place according to standard methods, e.g., as described in “The Quia Expressionist®”, 5th Edition, June 2003.

The above-described transformed host cells, which express the polypeptide according to the invention, are particularly well-suited for carrying the processes described below for the identification of PDE11-modulators in a cellular assay. In addition, it can be advantageous to immobilize the corresponding host cells on solid carriers and/or carryout a corresponding screening process on a high-throughput scale (high-through-put-screening).

All of the aforementioned nucleic acid sequences may be manufactured by being cut out of known nucleic acid sequences using methods such as enzymatic methods known to a person skilled in the art and recombined with known nucleic acid sequences. Moreover, all of the aforementioned nucleic acids may be, in a method known in the art, manufactured by chemical synthesis from the nucleotide building blocks, e.g., by fragment condensation of individual overlapping complementary nucleic acid building blocks of the double helix. For example, chemical synthesis of oligonucleotides may take place according to the known phosphoramidite method (Voet, Voet, 2nd Edition, Wiley Press, New York, pp. 896-897). The accumulations of synthetic oligonucleotides and filling of gaps using the Klenow fragment of DNA polymerase and ligation reactions, as well as general cloning processes, are described in Sambrook et al. (1989), Molecular Cloning: A Laboratory Manual, Cold Spring Harbor Laboratory Press.

The invention also concerns a process for the identification of a modulator of a human phosphodiesterase 11 (PDE11) comprising the following steps:

(a) Bringing a possible modulator of a human phosphodiesterase 11 (PDE11) into contact with a polypeptide according to the invention and

(b) Determination whether the possible modulator changes the adenylate cyclase activity of the polypeptide according to the invention compared to when the possible modulator is not present.

In a preferred embodiment of the process according to the invention, in step (a), in addition to the possible modulator of a human phosphodiesterase 11 (PDE11), cGMP is brought into contact with a polypeptide according to the invention.

In the process according to the invention, the possible PDE11 modulator, preferably in vitro with the preferably purified polypeptide according to the invention, and particularly preferably incubated with cGMP, and the change in adenylate cyclase activity of the polypeptide according to the invention compared to a test mixture without PDE11 modulator is measured.

Alternatively, the change in adenylate cyclase activity after addition of the possible PDE11 modulator to a test mixture containing the polypeptide according to the invention and possibly cGMP as well, may be measured. As described in greater detail below, the adenylate cyclase activity of the PDE11/CyaB1-chimera is determined by converting a specified amount of ATP into cAMP.

The modulator of a human phosphodiesterase 11 (PDE11), also referred to in the following as PDE11-modulator, refers to a substance that is capable, via binding to the GAF domains of PDE11, of modulating PDE11 activity, i.e., changing this activity, measured in this case with respect to the change in adenylate cyclase activity. Thus a PDE11 modulator acts via the allosteric centre of PDE11 and not or not only via the catalytic centre of PDE11. The modulator may be an agonist, in that it increases the enzymatic activity of PDE11 (PDE11 agonist) or an antagonist, in that it lowers the enzymatic activity of PDE11 (PDE11 antagonist).

For example, it was possible to show, surprisingly, using the process according to the invention, as described below, that cGMP constitutes a PDE11 agonist.

Preferred PDE11 modulators are also e.g., peptides, peptidomimetics, proteins, particularly antibodies, particularly monoclonal antibodies directed against GAF domains, amino acids, amino acid analogs, nucleotides, nucleotide analogs, polynucleotides, particularly oligonucleotides, and particularly preferred, so-called “small molecules” or SMOLs. Preferred SMOLs are organic or inorganic compounds, including heteroorganic compounds or organometallic compounds having a molecular weight smaller than 1,000 g/mol, particularly with a molecular weight of 200 to 800 g/mol, and particularly preferably with a molecular weight of 300 to 600 g/mol.

According to the present invention, a PDE11 modulator preferentially binds to the GAF domains in the polypeptides according to the invention (PDE11/CyaB1-chimera) and leads either directly to a change in the adenylate cyclase activity of the polypeptide according to the invention (PDE11/CyaB1-chimera) or to a change in the adenylate cyclase activity of the PDE11/CyaB1-chimera by the suppression of cGMP by PDE11/CyaB1-chimera.

If the method according to the invention is carried out only with cGMP or cAMP and without a PDE11 modulator as the substance to be tested, one obtains the dose-effect curve shown in FIG. 5. The PDE11A4/CyaB1-chimera is activated some 4-fold by 1 mM of cGMP. This corresponds to a % basal value of 400 and demonstrates that cGMP is a PDE11A4-GAF agonist. cAMP does not activate at 1 mM and has a % basal value of approx. 150, i.e., it is neither a GAF agonist nor an antagonist.

The modulation, i.e., the change, that is the increase or decrease in adenylate cyclase activity through the PDE11 modulator in a test mixture without cGMP is calculated as a % basal value according to the following formula:

$$\% Basal value = 100 \times [ \frac{Conversion with substance}{Conversion without substance} ]$$

If the % basal value in use of 100 μM of the possible PDE11 modulator is less than 50, this indicates a PDE11 antagonist that binds to the GAF domains in the PDE11/CyaB1-chimera, while a % basal value greater than 200 indicates a PDE11 agonist.

The invention therefore concerns a particularly preferred process according to the invention according to which, in the presence of the modulator, a decrease in adenylate cyclase activity is measured compared to absence of the modulator, and the modulator constitutes a PDE11 antagonist.

Moreover, the invention concerns a particularly preferred process according to the invention in which, when the modulator is present, an increase in adenylate cyclase activity is measured in comparison to the absence of the modulator and the modulator constitutes a PDE11 agonist.

In a particularly preferred embodiment of the process according to the invention, determination of adenylate cyclase activity takes place via measurement of the conversion of radioactively or fluorescently labeled ATP.

The measurement of adenylate cyclase activity of the polypeptide according to the invention, the PDE11/CyaB1-chimera, may take place via measurement of the conversion of radioactive [α-³²P]-ATP to [α-³²P]-cAMP.

Generally speaking, adenylate cyclase activity can be easily determined by measuring the resulting cAMP under antibody formation. There are various commercial assay kits for this purpose, such as the cAMP [³H-] or [¹²⁵-I] BioTrak® cAMP SPA-Assay from Amersham® or the AlphaScreen® or Lance® cAMP Assay from PerkinElmer®: they are all based on the principle that during the AC reaction, unlabeled cAMP originates from ATP. This competes with exogenously added 3H-, 125I-, or Biotin-labeled cAMP for binding to a cAMP-specific antibody. In the non-radioactive Lance® Assay, Alexa®-Flour and the antibodies are bound, which with the tracer produces a TR-FRET signal at 665 nm. The more unlabeled cAMP is bound, the weaker the signal triggered by the labeled cAMP. With a standard curve, the signal intensities of the corresponding cAMP concentration can be classified.

Analogously to the High-Efficiency Fluorescence Polarization (HEFP™)-PDE Assay from Molecular Devices, which is based on IMAP technology, one may use fluorescence-labeled substrate instead of radioactively labeled substrate. In the HEFP-PDE Assay, fluorescein-labeled cAMP (Fl-cAMP) is used, which is converted by the PDE to fluorescein-labeled 5′ AMP (Fl-AMP). The Fl-AMP selectively binds to special beads, causing the fluorescence to be strongly polarized. Fl-AMP does not bind to the beads, so that an increase in polarization of the amount of Fl-AMP produced is proportional. For a corresponding AC-test, fluorescein-labeled ATP instead of Fl-cAMP and beads, which bind selectively to Fl-cAMP instead of Fl-cAMP (e.g., beads loaded with cAMP antibodies), may be used.

In a further preferred embodiment of the process according to the invention, in order to differentiate whether the changed % basal value is caused by an effect of the substance modulated by GAF or by direct modulation of the AC catalytic centre, an additional counter screen is carried out.

Therefore, the invention also concerns a preferred process according to the invention in which, in order to exclude direct modulators of the catalytic domains of adenylate cyclase, a process according to the invention is carried out using a polypeptide that has the catalytic domain of an adenylate cyclase and shows no functional GAF domain of a human phosphodiesterase 11 (PDE11).

Preferably, the % basal value is also determined analogously to the above-described process, preferably with a protein rather than the PDE11/CyaB1-chimera, which preferably only

(a) contains the AC catalytic centre or

(b) contains mutations on the amino acids essential for the GAF function, or

(c) the N-terminus is shortened by the GAF domain.

An example of a) is a polypeptide with the amino acid sequence SEQ. I.D. NO. 1, provided that N-terminal A2 through L775 are lacking.

An example of b) is a polypeptide with the amino acid sequence SEQ. I.D. NO. 1, provided that it contains the mutation D355A.

An example of c) is polypeptide with the amino acid sequence SEQ. I.D. NO. 1, provided that the partial sequence from L240 to K568 is lacking.

If 100 μM of a substance with the protein modified according to a, b, or c has a % basal value of less than 50, there is inhibition of the AC catalytic centre, and pure GAF antagonism can be ruled out.

In a further preferred embodiment of the process according to the invention, the process is carried out as a cellular assay in the presence of an above-described host cell according to the invention.

In addition, the cAMP produced, as a measure of adenylate cyclase activity, may also be determined in cellular assays, such as described in Johnston, P. Cellular assays in HTS, Methods Mol Biol. 190, 107-16 (2002) and Johnston, P. A.: Cellular platforms for HTS, three case studies. Drug Discov Today, 7, 353-63 (2002).

In addition, cDNA of the polypeptides according to the invention, the PDE11/CyaB1-chimera, is preferably introduced via suitable interfaces into a transfection vector and transfected with the resulting vector construct of suitable cells, such as CHO or HEK293-cells. The cell clones that express the polypeptide according to the invention in a stable manner are selected.

The intracellular cAMP level of the transfected cell clones is considerably affected by the adenylate cyclase activity of the polypeptides according to the invention. By inhibiting adenylate cyclase activity, GAF antagonists cause a reduction and GAF agonists an increase in intracellular cAMP.

The amount of cAMP can either be measured following lysis of the cells by the above-described methods (BioTrak®, AlphaScreen®, or HEFP®), or directly in the cells. For this purpose, a reporter gene in the cell line is preferably coupled to a CRE (cAMP response element) (Johnston, P. Cellular assays in HTS, Methods Mol Biol. 190, 107-16 (2002)). An elevated cAMP level leads to increased binding of CREB (cAMP response element binding protein) to the CRE regulator and therefore to elevated transcription of the reporter gene. As a reporter gene, for example, one may use Green Fluorescent Protein, β-galactosidase or luciferase, the expression levels of which may be determined by fluorometric, photometric, or luminometric methods, as in Greer, L. F. and Szalay, A. A. Imaging of light emission from the expression of luciferase in living cells and organisms, a review. Luminescence 17, 43-72 (2002) or Hill, S. et al. Reporter-gene systems for the study of G-protein coupled receptors. Curr. Opin. Pharmacol. 1, 526-532 (2001).

In a particularly preferred embodiment, the above-described process according to the invention is used, specifically as a cellular assay, in high-throughput scale.

The following examples illustrate the present invention, but without restricting it to said examples:

### EXAMPLE 1

### Manufacturing of Recombinant DNA Coding for a PDE11/CyaB1-Chimera

Cloning was carried out according to the standard method. The original clone with the gene for human PDE11A4 (Genbank Accession No. BAB62712) was provided in a vector. By means of PCR, cloning of the PDE2-GAF chimera was carried out in a manner similar to that described by Kanacher et al., EMBO J. 2002. With specific primers, a gene fragment hPDE11A4₁₋₃₉₁ was amplified which coded for the PDE11A4-N-terminal with the GAF-A domain and contains the N-terminal of a BglII and C-terminal of a Xbal interface. Analogously, a gene fragment hPDE11A4₃₉₂₋₅₆₉, which codes for the GAF-B domain and contains the N-terminal of a Xbal interface and C-terminal of a SalI interface was amplified. The two fragments were joined via the Xbal interface to hPDE11A4₁₋₅₆₉ via subcloning steps in the cloning vector pBluescriptlI SK(−). On the gene fragment hPDE11A4₁₋₅₆₉, a gene fragment CyaB1₃₈₆₋₈₅₉ generated by PCR was attached to the catalytic domain of adenylate cyclase CyaB1 (Genbank Accession No. D89623) via the SalI interface C-terminal. In this case, the N-terminal SalI interface of hPDE11A4₁₋₅₆₉ was cloned on the C-terminal Xhol interface of CyaB1₃₈₆₋₈₅₉ and L386 was mutated from CyaB1 to V. All cloning steps took place in E. coli XI1blueMRF.

The gene for the PDE11-GAF chimera was recloned in the expression vector pQE30 (from Quiagen).

### EXAMPLE 2

### Expression and Purification of the Polypeptide

The pQE30 vector with a gene for the PDE11-GAF chimera was retransformed in E. coli BL21 cells. The expression and purification of the protein took place as described in “The QiaExpressionist®”, 5th Edition, June 2003. In this case, the optimal protein yield under the expression conditions of induction with 25 μM IPTG, 16 hour incubation at 16° C., and subsequent French Press Treatment of E. coli, was achieved.

### EXAMPLE 3

### Conduct of Assays

The adenylate cyclase activity of the PDE11A4/CyaB1-chimera is measured with and without the test substance. In this case, the adenylate cyclase activity or conversion of a specified amount of ATP to cAMP and its chromatographic separation over two columns steps may be determined according to Salomon et al. To detect conversion, [α-³²P]-ATP was used as a radioactive tracer, and the amount of [α-³²P]-cAMP produced was measured. ³H-cAMP is used as an internal standard for a recovery rate. The incubation time should be between 1 and 120 min, the incubation temperature between 20 and 45° C., the Mg²⁺-cofactor concentration between 1 and 20 mM (corresponding amounts of Mn²⁺ may also be used as a cofactor) and the ATP concentration between 0.5 μM and 5 mM. An increase in the conversion with the substance compared to without the substance indicates a GAF-agonistic effect. If conversion is inhibited by adding the substance, this indicates a GAF-antagonistic effect of the substance. A GAF antagonism can also be measured via blockage of activation of PDE11A4/CyaB1-chimera by the native GAF ligand cGAP. In addition, the conversion at rising cGAP concentration is measured with and without the substance. If the conversion rates with the substance are below those without the substance, this indicates GAF antagonism of the substance.

A reaction test contains the following:
50 μL AC-test-cocktail (glycerol 43.5% (V/V), 0.1 M tris/HCl, pH 7.5, 20 mM Mg Cl₂)
40-x μL enzyme dilution (depending on activity, contains 0.1-0.3 μg of PDE10/CyaB1-chimera in 0.1% (W/V) aqueous BSA solution)
x μL substance
10 μL 750 μM ATP-start solution, incl. 16-30 kBq [α-³²P]-ATP.

The protein samples and the cocktail are measured in 1.5 mL reaction containers on ice, the reaction with ATP is started, and incubation is carried for 10 minutes at 37° C. The reaction is stopped with 150 μL of AC stop buffer, the reaction vessels are placed on ice, and 10 μL 20 mM cAMP incl. 100 Bq [2,8-³H]-cAMP and 750 μL of water were added.

Each test mixture is carried in duplicate. As a blank, a test mixture with water instead of enzyme was used. With a test mixture without substance and cGMP, the basal enzyme activity is determined. In order to separate the ATP and cAMP activity, each sample is run on glass tubes with 1.2 g Dowex-50WX4-400, and after it sinks in, it is washed with 3-4 mL of water. After this, 5 mL of water was used to elute the aluminum oxide columns (9×1 cm glass columns with 0.5 g Al₂O₃ 90 active, neutral) and this was eluted with 4 mL of 0.1 M tris/HCl, pH 7.5 in a scintillation container with 4 mL of prepared scintillator Ultima XR Gold. After thoroughly mixing, counting was carried out using a liquid scintillation counter. The amounts of radioactively labeled cAMP and ATP used are directly counted as ³H and ³P totals directly in 5 mL of elution buffer and 4 mL scintillator.

The conversion again is calculated as enzyme activity in the following formula:

$$A [ \frac{p mol [ cAMP ]}{mg [ Protein ] \times min} ] = \frac{Substrate [ μM ]}{Time [ min ]} \times \frac{{10}^{5}}{Protein amount [ μg ]} \times \frac{{cpm [ {}^{32} P ]}_{sample} - {cpm [ {}^{32} P ]}_{Leerwert}}{{cpm [ {}^{32} P ]}_{total}} \times \frac{{cpm [ {}^{3} H ]}_{total}}{{cpm [ {}^{3} H ]}_{sample} - 3 {\% [ {}^{32} P ]}_{sample}}$$

The inhibition or activation of the enzyme by the substance is calculated as % basal value according to the following formula:

$$\% Basal value = 100 \times [ \frac{conversion with substance}{conversion without substance} ]$$

If the % basal value for 100 μM of the substance is less than 50, this indicates, excluding inhibition of the AC-catalytic centre, a GAF antagonist, while a % basal value of greater than 200 indicates GAF agonists.

In a test mixture with 100 μM of cGMP, a GAF antagonist is present if the % basal value in use of 100 μM of the substance to be tested is less than 90.

The columns were regenerated as follows after use:

Dowex columns: 5 mL 2N HCl, 2×5 mL water

Aluminum oxide columns: 2×5 mL 0.1 M tris/HCl, pH 7.5

### DESCRIPTION OF THE FIGURES

FIG. 1: Amino acid sequence of PDE11/CyaB1-chimera

FIG. 2: cDNA sequence of PDE11/CyaB1-chimera

FIG. 3: Protein sequence of PDE11/CyaB1-chimera after purification. Italics=purification day from the expression vector (pQE30 from Quiagen); bold=N-terminal with PDE11-GAF domains; bold and underlined=GAF_{A} domain and GAF_{B} domain; V386 was mutated from L386 for insertion of the cloning interface; underlined=C-terminal of CyaB1 with catalytic domain

FIG. 4: Schematic drawing of chimeric PDE11/CyaB1 polypeptide

FIG. 5: Activation of PDE11/CyaB1-chimera through cyclic nucleotides When the Assay is carried out with cGMP or cAMP as the substance to be tested, this yields the dose-effect curve shown in FIG. 5. The PDE11A4/CyaB1-chimera is activated approximately 4-fold by 1 mM of cGMP. This corresponds to a % basal value of 400 and shows that cGMP is a PDE11A4-GAF agonist. cAMP does not activate at 1 mM and has a % basal value of approx. 150, which means that it is neither a GAF agonist nor an antagonist.

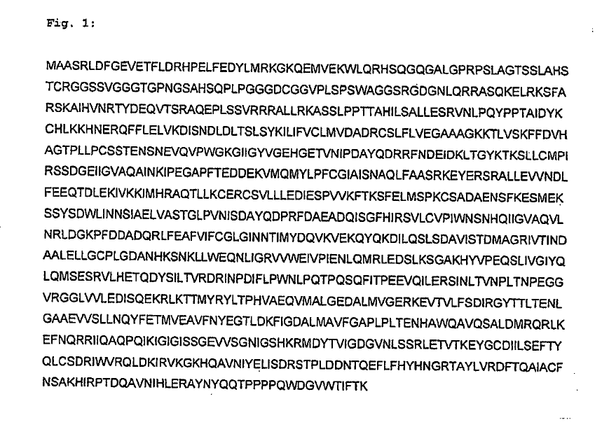

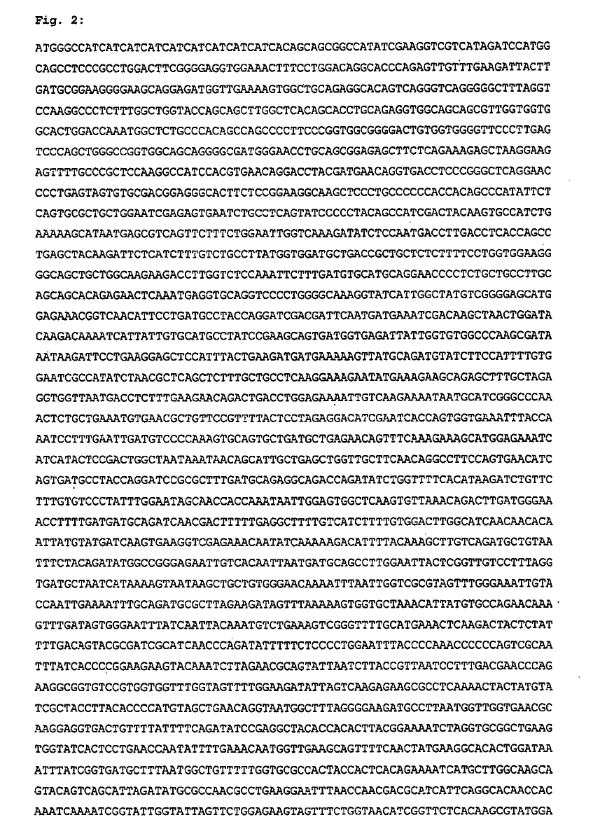

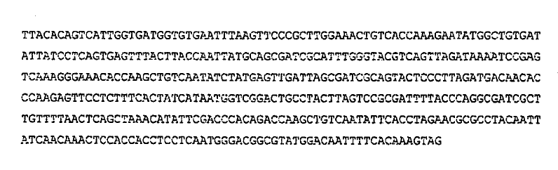

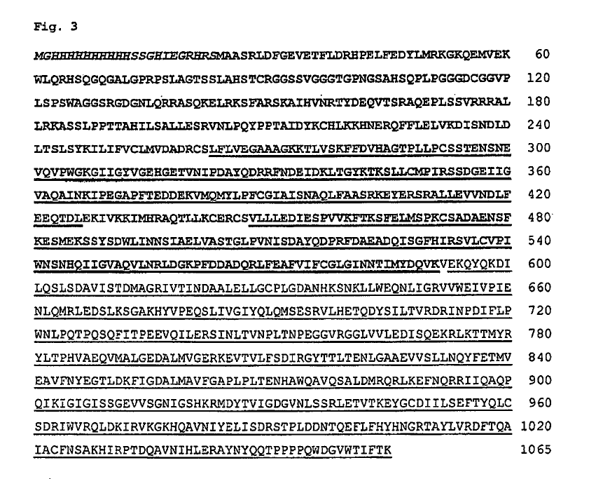

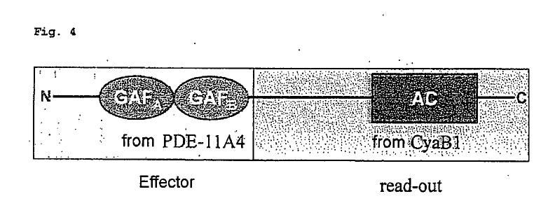

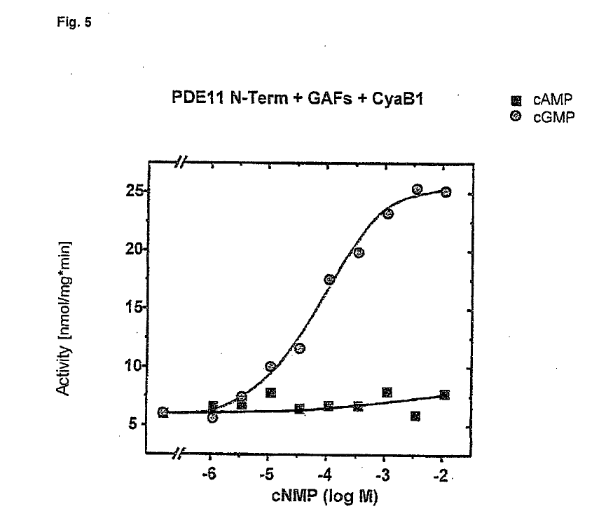

1. A polypeptide, comprising, functionally linked:
(a) a GAF_{A} domain and GAF_{B} domain of a human phosphodiesterase 11 (PDE11) or its functionally equivalent variants and
(b) a catalytic domain of an adenylate cyclase or its functionally equivalent variants.

2. The polypeptide according to claim 1, characterized in that the phosphodiesterase 11 (PDE11) is selected from the group consisting of PDE11A1, PDE11A2, PDE11A3, PDE11A4, and their respective functionally equivalent variants.

3. The polypeptide according to claim 1, characterized in that the phosphodiesterase 11 (PDE11) has the isoform PDE11A4.

4. The polypeptide according to claim 1, characterized in that the GAF_{A} domain shows an amino acid sequence containing the amino acid sequence SEQ. I.D. NO. 6 or a sequence derived from this sequence by substitution, insertion, or deletion of amino acids, which has an identity of at least 90% at the amino acid level with the sequence SEQ. I.D. NO. 6 and shows the property of a GAF_{A} domain.

5. The polypeptide according to claim 4, characterized in that the GAF_{A} domain has an amino acid sequence containing the amino acid sequence SEQ. I.D. NO. 6.

6. The polypeptide according to claim 1, characterized in that the GAF_{B} domain has an amino acid sequence containing the amino acid sequence SEQ. I.D. NO. 8 or a sequence derived from this sequence by substitution, insertion, or deletion of amino acids, which has an identity of at least 90% on an amino acid level with the sequence SEQ. I.D. NO. 8 and has the property of a GAF_{B} domain.

7. The polypeptide according to claim 6, characterized in that the GAF_{B} domain has an amino acid sequence containing the amino acid sequence SEQ. I.D. NO. 8.

8. The polypeptide according to claim 1, characterized in that the functionally linked GAF_{A} domain and GAF_{B} domain of a human phosphodiesterase 11 (PDE11) or its functionally equivalent variants have an amino acid sequence containing the amino acid sequence SEQ. I.D. NO. 10 or a sequence derived from this sequence by substitution, insertion, or deletion of amino acids, which has an identity of at least 70% on an amino acid basis with the sequence SEQ. I.D. NO. 10 and shows the regulatory property of the GAF domain of a human phosphodiesterase 11 (PDE11), in which the obtained amino acid sequences of the GAF_{A} domain, SEQ. I.D. NO. 6, and the GAF_{B} domain, SEQ. I.D. NO. 8, vary by a maximum of 10% through substitution, insertion, or deletion of amino acids.

9. The polypeptide according to claim 1, characterized in that the functionally linked GAF_{A} domain and GAF_{B} domain of a human phosphodiesterase 11 (PDE11) or their functionally equivalent variants show an amino acid sequence selected from the group consisting of
(a) N-terminus of human PDE11A4 from amino acid M24 up to amino acid K591, and
(b) SEQ. I.D. NO. 10.

10. The polypeptide according to claim 1, characterized in that the adenylate cyclase constitutes an adenylate cyclase of bacterial origin containing a GAF domain or its respective functionally equivalent variants.

11. The polypeptide according to claim 1, characterized in that the adenylate cyclase constitutes an adenylate cyclase selected from the group consisting of
(a) adenylate cyclase from Anabaena sp. PCC 7120 or its functionally equivalent variants,
(b) adenylate cyclase from Anabaena variabili ATTC 29413 or its functionally equivalent variants,
(c) adenylate cyclase from Nostoc punctiforme PCC 73102 or its functionally equivalent variants,
(d) adenylate cyclase from Trichodesmium erythraeum IMS 101 or its functionally equivalent variants,
(e) adenylate cyclase from Bdellovibrio bacteriovorus HD 100 or its functionally equivalent variants, and
(f) adenylate cyclase from Magnetococcus sp. MC-1 or its functionally equivalent variants.

12. The polypeptide according to claim 1, characterized in that the adenylate cyclase constitutes an adenylate cyclase from Anabaena sp. PCC 7120 of the isoform CyaB1 or CyaB2 or its functionally equivalent variants.

13. The polypeptide according to claim 1, characterized in that the catalytic domains of an adenylate cyclase or its functionally equivalent variants show an amino acid sequence containing the amino acid sequence SEQ. I.D. NO. 12 or a sequence derived from this sequence by substitution, insertion, or deletion of amino acids, which has an identity of at least 90% on an amino acid basis with the sequence SEQ. I.D. NO. 12 and shows the catalytic property of an adenylate cyclase.

14. The polypeptide according to claim 1, characterized in that the catalytic domain of an adenylate cyclase or its functionally equivalent variants shows an amino acid sequence selected from the group consisting of
(a) C-terminus of CyaB1 of the amino acids L386 through K859, in which L386 is replaced by CyaB1 through V386, and
(b) SEQ. I.D. NO. 12.

15. The polypeptide according to claim 1, containing the amino acid sequence SEQ. I.D. NO. 1 or SEQ. I.D. NO. 4 or a sequence derived from these sequences through substitution, insertion, or deletion of amino acids which has an identity of at least 70% on an amino acid basis with the sequence SEQ. I.D. NO. 1 or 4 and the regulatory properties of the GAF domain of a human phosphodiesterase 11 (PDE11) and the catalytic properties of an adenylate cyclase, wherein the obtained amino acid sequences of the GAF_{A} domain, SEQ. I.D. NO. 6, the GAF_{B} domain, SEQ. I.D. NO. 8, and the catalytic domain of adenylate cyclase, SEQ. I.D. NO. 12, vary by a maximum of 10% through substitution, insertion, or deletion.

16. The polypeptide according to claim 1, including the amino acid sequence SEQ. I.D. NO. 1 or SEQ. I.D. NO. 4.

17. A polypeptide with the amino acid sequence SEQ. I.D. NO. 1 or SEQ. I.D. NO. 4.

18. A polynucleotide coding for one of the polypeptides according to claim 1.

19. The polynucleotide according to claim 18, containing as partial sequences
(a) SEQ. I.D. NO. 5 or a nucleic acid sequence that hybridizes with the nucleic acid sequence SEQ. I.D. NO. 5 under stringent conditions, and
(b) SEQ. I.D. NO. 7 or a nucleic acid sequence that hybridizes with the nucleic acid sequence SEQ. I.D. NO. 7 under stringent conditions, and
(c) SEQ. I.D. NO. 11 or a nucleic acid sequence that hybridizes with the nucleic acid sequence SEQ. I.D. NO. 11 under stringent conditions.

20. A polynucleotide containing the nucleic acid sequence SEQ. I.D. NO. 2.

21. A polynucleotide of the nucleic acid sequence SEQ. I.D. NO. 2.

22. A recombinant plasmid vector containing a polynucleotide according to claim 18.

23. A recombinant host cell containing a plasmid vector according to claim 22.

24. A process for the manufacture of a polypeptide which comprises, functionally linked:
(a) a GAF_{A} domain and GAF_{B} domain of a human phosphodiesterase 11 (PDE11) or its functionally equivalent variants and
(b) a catalytic domain of an adenylate cyclase or its functionally equivalent variants, by culturing a recombinant host cell according to claim 23, expression and isolation of said polypeptide.

25. A process for the identification of a modulator of a human phosphodiesterase 11 (PDE11) comprising the steps
(a) bringing a possible modulator of a human phosphodiesterase 11 (PDE11) into contact with a polypeptide according to claim 1 and
(b) determination of whether the possible modulator modifies the adenylate cyclase activity of the polypeptide according to claim 1 compared to when the possible modulator is absent.

26. The process according to claim 25, wherein, in step (a) in addition to the possible modulator, a human phosphodiesterase 11 (PDE11) cGMP is brought into contact with the polypeptide.

27. The process according to claim 25, characterized in that the determination of the adenylate cyclase activity takes place via measurement of the conversion of radioactively or fluorescently labeled ATP.

28. The process according to claim 25, characterized in that a decrease in adenylate cyclase activity is measured in the presence of the modulator compared to when the modulator is absent, and the modulator thus constitutes a PDE11 antagonist.

29. The process according to claim 25, characterized in that an increase in adenylate cyclase activity is measured in the presence of the modulator compared to when the modulator is absent, and the modulator thus constitutes a PDE11 agonist.

30. The process according to claim 25, characterized in that, in order to exclude direct modulators of the catalytic domain of adenylate cyclase, a process according to claim 25 is carried out using a polypeptide that shows the catalytic domain of an adenylate cyclase and shows no functional GAF domain of a human phosphodiesterase 11 (PDE11).

31. A process for the identification of a modulator of a human phosphodiesterase 11 (PDE11) comprising the steps
(a) bringing a possible modulator of a human phosphodiesterase 11 (PDE11) into contact with a polypeptide, comprising, functionally linked:
(i) a GAF_{A} domain and GAF_{B} domain of a human phosphodiesterase 11 (PDE11) or its functionally equivalent variants and
(ii) a catalytic domain of an adenylate cyclase or its functionally equivalent variants and
(b) determination of whether the possible modulator modifies the adenylate cyclase activity of said polypeptide compared to when the possible modulator is absent, wherein the process is carried out as a cellular assay in the presence of a host cell according to claim 23.

32. The process according to claim 31, characterized in that the process is used on a high-throughput scale.
